(19) United States
(12) Patent Application Publication
(10) Pub. No.: US 2003/0159182 A1
(43) Pub. Date: Aug. 21, 2003

(54) Production of therapeutic proteins in transgenic cereal crops

(76) Inventors: Eilleen Tackaberry, Ottawa (CA); Anil Dudani, Ottawa (CA); Peter Ganz, Orleans (CA); Ravinder Sardana, Ottawa (CA); Illimar Altosaar, Ottawa (CA)

Correspondence Address: Mr. W. Charles Kent, Ridout & Maybee LLP, 19th Floor, 150 Metcalfe St., Ottawa, ON, K2P 1P1, CA

(21) Appl. No.: 10/230,365

(22) Filed: Aug. 29, 2002

Related U.S. Application Data
(60) Provisional application No. 60/315,363, filed on Aug. 29, 2001.

(51) Int. Cl. A61K 39/12; A01H 1/00; C12N 15/82

(52) U.S. Cl. 800/288; 424/186.1

(57) ABSTRACT

There is provided a herpes virus vaccine produced in in the seeds of a cereal crop and a method of producing the vaccine. The method comprises: a) obtaining a nucleic acid sequence encoding a herpes virus antigen; b) introducing the nucleic acid sequence into cereal plant tissue competent to form seeds; c) permitting said cereal plant tissue to develop; and, d) directing preferential expression of the antigen encoded by the nucleic acid sequence in seeds formed by the cereal plant tissue. Herpes viruses antigens of particular interest include all or antigenic portions of gB (from human cytomegalovirus (“HCMV”)), gH (from HCMV), and gD (from herpes simplex virus 1 or 2), as well as antigens from Epstein Barr virus and varicello-zoster virus-8. Envelope glycoproteins from herpes viruses are antigens of interest. Cereal crops of particular interest include rice, wheat, oats, barley, and corn. Vaccines produced according to the invention are very stable and may be administered by a variety of routes, including injection and contact with mucosal membranes, such as by oral administration in purified or unpurified form.

### BACKGROUND OF THE INVENTION

Transgenic plants offer many potential advantages over conventional expression systems for the production of recombinant subunit vaccines. These include the ability to effect post-translational modifications, a low risk of contamination by organisms pathogenic to humans, and simple, inexpensive scale-up for commercial production. Furthermore, plants or plant products which are edible provide the additional unique opportunity of serving as vehicles for oral delivery of the vaccine. Despite efforts to develop such plant-derived vaccines, biochemical and immunological challenges have limited success to date. (See for example, Moffat et al., Science 1995, 268, 658-660; Kusnadi et al., Biotech. and Bioengin. 1997, 56, 473-484; Mason et al., Proc. Natl. Acad. Sci. USA 1996, 93, 5335-5340; Mason et al., Vaccine 1998, 16, 1336-1348; Thanavala et al., Proc. Natl. Acad. Sci. USA 1995, 92,3358-3361; and, Tacket et al., Nature Med. 1998, 4,607-609.)

Human cytomegalovirus (“HCMV”) is a widely distributed member of the herpes virus family that is transmitted by blood and other body secretions. In immunocompromised individuals such as AIDS patients, organ transplant recipients and low weight pre-term infants, the virus can cause severe and/or lethal disease, while congenital infection may result in damage to the central nervous system. The HCMV encoded glycoprotein B complex (“gB”) is a transmembrane protein of 907 amino acids (for the prototype Towne strain) which is initially synthesized in infected cells as a 105 kDa non-glycosylated polypeptide. In normal infected mammalian host cells, the gB polypeptide undergoes post-translational glycosylation, cleavage of the N-terminal 24 amino acid signal peptide, oligomerization and folding which take place in the endoplasmic reticulum of the cell, where it is transiently associated with a membrane-bound chaperonin. This results in transport of a 150 kDa gB precursor to the Golgi complex where further carbohydrate modifications occur and the polypeptide is proteolytically cleaved to yield products of 116 kDa and 58 kDa which are disulfide linked. Both species are targets for neutralizing and non-neutralizing antibodies, each representing both continuous and discontinuous epitopes. A phosphorylation site is located in the cytoplasmic tail and may be important for correct intracellular trafficking. The sequence of gB (Towne) is reported in Spaete et al., Virology 167(1), 207 (1988), Pub. Med. Acc. No. M22343.

Mammalian immune responses are highly specific and sensitive to even minor differences between potential antigenic sites. Thus, changes to the post-translational modification of an antigen such as gB will have the potential to render it unsuitable for use as a vaccine against infection by the native organism.

Plant seeds are an ideal organ for the targeted synthesis of heterologous proteins. However, where the proteins of interest are of non-plant origin, numerous technical challenges arise in the production and recovery of useful transgenic proteins. In particular, differences in post-translational modification and transport may render plant-produced proteins unsuitable for some uses in mammals.

A number of specific promoters of seed protein genes have been identified and characterized. For example, glutelin (Gt), which represents the major reserve endosperm protein in rice seeds, is encoded by a small multigene family with subfamilies designated Gt1, Gt2, Gt3, etc. The glutelin promoters have been shown to direct the expression of various reporter genes in transgenic plant systems, resulting in gene expression that is tissue specific and developmentally regulated.

The expression of a recombinant gB construct in tobacco seed was reported by Tackaberry et al. (Vaccine 17: 3020 (1999)). Although the recombinant gB reported was recognized in vitro by monoclonal antibodies to naturally occurring gB, no ability of the recombinant gB to induce a specific immune response in vivo (as required for function as a vaccine) was shown. Moreover, tobacco seeds are not a preferred vehicle for vaccine production. For example, as tobacco seeds are not generally a major food substance in a mammalian diet, they are not well suited to the production of oral vaccines.

Thus, it is an object of the present invention to provide a method for the production of a viral antigen in plant material which is a major food substance in a mammalian diet.

### SUMMARY OF THE INVENTION

In an embodiment of the invention there is provided a method of producing a herpes virus vaccine in the seeds of a cereal crop. The method comprises: a) obtaining a nucleic acid sequence encoding a herpes virus antigen; b) introducing the nucleic acid sequence into cereal plant tissue competent to form seeds; c) permitting said cereal plant tissue to develop; and, d) directing preferential expression of the antigen encoded by the nucleic acid sequence in seeds formed by the cereal plant tissue.

In an embodiment of the invention there is provided a cereal crop containing a herpes virus antigen.

In an embodiment of the invention there is provided a method of inducing an immune response in a mammal which response reduces the risk of subsequent infection of the mammal by a herpes virus. The method comprises administering to the mammal a herpes virus antigen produced in a cereal crop.

In an embodiment of the invention there is provided a nucleic acid encoding: a signal peptide sequence directed to the endoplasmic reticulum, then the Golgi Apparatus, and then to protein storage vacuoles; a herpes virus antigen; and, a promoter sequence allowing preferential expression of a controlled gene in cereal crop seeds, wherein the portion of the nucleic acid encoding the signal sequence and the herpes virus antigen is the controlled gene.

In an embodiment of the invention there is provided a cereal crop cell containing a stably incorporated nucleic acid encoding an antigenic domain of a herpes virus antigen.

### BRIEF DESCRIPTION OF THE DRAWINGS

These and other advantages of the invention will become apparent upon reading the following detailed description and upon referring to the drawings in which:—

FIG. 1(a) is a diagrammatic representation of the insert structure of plasmid vectors pPH1 and pPH2.

FIG. 1(b) is a diagrammatic representation of the insert structure of plasmid vectors pSVL/CMVgB and pPH2/gB.

FIG. 1(c) is a diagrammatic representation of the insert structure of plasmid vector pPH2/gB/NOS-T.

FIG. 1(d) is a diagrammatic representation of the insert structure of plasmid vectors pKHG4 and pKHG4/pPH2/gB/NOS-T.

FIG. 2 is a photographic depiction of the results of Example 3.

FIG. 3 is a photographic depiction of the results of Example 4.

FIG. 4 is a graphical representation of the results of Example 6.

FIG. 5 is a photographic depiction of the results of Example 7.

While the invention will be described in conjunction with the illustrated embodiments, it will be understood that it is not intended to limit the invention to such embodiments. On the contrary, it is intended to cover all alternatives, modifications and equivalents as may be included within the spirit and scope of the invention as defined by the appended claims.

### DETAILED DESCRIPTION OF THE PREFERRED EMBODIMENTS

There is provided a method for producing viral antigens in rice and other cereal crops. Of particular interest are antigens for herpes viruses. As rice and other cereal crops are major food substances in the diet of many mammals, the viral antigens produced therein are useful, without further purification, as oral vaccines having oral immunogenicity against corresponding viruses. In addition, expression of the antigen in seeds allows ready separation of the plant tissues of interest for purification or other purposes. Allergic reactions to some cereal crops such as rice are extremely rare, further increasing their usefulness for vaccine production.

Cereal crops of particular interest include rice, wheat, oats and barley. In some instances, cereal crops which are graminoids or non-graminoids of the family Poaceae will be desired. In some instances cereal crops from the Avena, Zea, Triticum, Secale or Hordeum will be desirable. In some instances oat, corn, common wheat, or Hordeum vulgare L. will be desired.

Previous work has shown that rice can be difficult to transform using Agrobacterium.

Surprisingly, present invention allows for the expression in cereal crops of an immunogenic herpes virus protein. To achieve this, the method of the invention overcomes challenges presented by a number of transmembrane virus antigens, including: large antigen size, extensive glycosylation and post-translational cleavage. Thus, in light of the disclosure herein, it is possible to produce a variety of viral antigens in monocotolydonous plants. Cereal plants such as rice, wheat, oats and barley will often be desired for the expression of viral antigens. Viral antigens of interest will often include virally encoded proteins which are either very large (for example over 150 or 200 amino acids in mature length), are transmembrane proteins and/or are modified extensively following translation. In some instances, the expression of subunit viral antigens will be desired.

In some instances it will be desired to express particular regions of a viral antigen in plant seeds. For example, expression of either gp116 or gp 55 (also referred to as gp58) (subunits of the gB protein ordinarily generated by post-translational cleavage) in rice provides viral antigen which can be employed as a HCMV vaccine in mammals (with or without enrichment or purification). Administration may in some instances be by injection or by contact with a mucous membrane (e.g. oral or nasal administration). Similarly, vectors including nucleic acids encoding known antigenic regions of viral antigens may be employed. For example, within gB, three major antigenic regions have been identified and nucleic acids encoding one or more such regions may be employed to produce a HCMV vaccine in cereal crops. Major antigenic regions of gB include: AD-1 (amino acids 552-635), AD-2 (amino acids 50-77), and AD-3 (amino acids 783-907) (numbers refer to the mature protein). The method of the present Invention allows for production and use of suitable constructs directing the expression in plant seeds of any one or a combination of these antigenic domains with or without expression of the naturally occurring intervening amino adds.

As used herein the term “herpes virus antigen” means an antigen which, when expressed in a normal mammalian patient, is capable of stimulating the production of specific antibodies which will recognize the corresponding naturally occurring antigen. In some instances, the mammalian patient is a human, a rabbit, a guinea pig, a bovine or a swine. Herpes viruses of particular interest include: cytomegaloviruses (including cytomegaloviruses specific for distinct species, such as human cytomegalovirus (HCMV), guinea pig cytomegalovirus, murine cytomegalovirus, simian cytomegalovirus, rat cytomegalovirus, African green monkey cytomegalovirus, Rhesus cytomegalovirus, baboon cytomegalovirus, porcine cytomegalovirus, mandrilus cytomegalovirus, chimpanzee cytomegalovirus, vervet cytomegalovirus macacca mulatta cytomegalovirus, bovine cytomegalovirus, canine cyomegalovirus, rabbit cytomegalovirus and feline cytomegalovirus), herpes simplex viruses types 1 and 2, Epstein Barr virus, and varicello-zoster virus-8. In some instances the herpes virus antigen is an envelope glycoprotein from a herpes virus. In some instances, the herpes virus antigen is at least one of gB (from HCMV), gH (from HCMV) and gD (from HSV 1 or 2). As used herein, the phrase “herpes virus antigen” includes full length herpes virus antigens and antigenic portions of herpes virus antigens corresponding to native antigenic regions which are accessible for specific recognition by antibodies when the full length native protein is produced during normal infection of a host

As used herein, the phrase “high levels of expression in seeds”, when referring to expression of the protein product of a gene, means expression of the protein product of the gene as at least about an average of 0.25% of total protein in the mature seed. In some instances the invention provides expression of the protein product of the gene at an average of at least about 0.5% of total protein in the mature seed. In some cases expression of at least an average of about 1% of total protein in the mature seed is provided. In some instances expression of at least an average of about 2% of total protein in the mature seed is provided.

As used herein, the phrase “preferential expression in seeds” when referring to gene expression means that the protein product of the gene is, on average, present in higher levels in mature seeds than in other portions of the mature plant. The present invention provides preferential expression of herpes virus antigen in the seeds of cereal crops. In some instances the invention provides average expression of the protein in the mature seed at least twice as high as In other portions of the mature plant. In some instances the invention provides average expression in mature seeds at least four times higher than in other portions of the mature plant. In some instances the invention provides average expression of the protein in mature seeds at least 10 times higher than in other portions of the mature plant.

As used herein, the term “seed promoter” refers to a gene promoter which either (or both) directs high levels of protein expression In seeds, or which directs preferential protein expression in seeds.

Herpes virus antigens produced in cereal crops are useful as vaccines against Infection of mammalian subjects by the corresponding native herpes virus. In light of the disclosure herein, one skilled In the art can readily identify an appropriate dose level and frequency of vaccine administration.

Where the vaccine is administered by injection of antigen (either purified, partly purified or in ground seed material), a total per-treatment antigen dose of between about 4 μg/kg and 10 mg/kg body weight will in some instances be desirable. In some instances it will be desirable to split each total per-treatment dose between two or more Injection sites. In some instances a combination of subcutaneous injection and intramuscular injection will be desirable. In some instances, a total per-treatment antigen dose of between about 200 μg and 5 mg/kg body weight will be desirable. In some instances a total per-treatment antigen dose of between about 250 μg/kg and 1 mg/kg body weight will be desirable.

In some instances a series of 2 to 5 treatments, administered about 1 week to 6 weeks apart will be desirable.

Where the vaccine is administered by inducing contact with one or more mucosal surface of the mammalian patient's body, a total per-treatment antigen dose of between about 50 μg/kg and 400 mg/kg body weight will in some instances be desirable.

Mucosal surfaces of interest include intestinal mucosal surfaces (including the lymphoid tissue of the small intestine), nasal mucosal surfaces, as well as rectal, vaginal, and conjunctival mucosal surfaces. Lower antigen doses will generally be employed where substantially direct application of the antigen to the mucosal surface is employed. (For example by nasal administration of the antigen in a finely dispersed nasal spray.) Where the actual effective dose of antigen reaching the mucosal surface is likely to be significantly reduced (as may occur by digestion of ground seeds following oral administration) higher antigen doses may be preferred.

Where the vaccine is administered nasally, an antigen dose of between about 50 μg/kg and 50 mg/kg body weight will in some instances be desired. In some instances, nasal administration of an antigen dose of between about 500 μg and 10 mg/kg body weight will be desired. When the vaccine is administered nasally, it will in some instances be desirable to enrich the antigen level (relative to other seed material) prior to nasal administration. In some instances the antigen will be present at levels at least about 50 times as high (by weight) as the level of dry seed material. In some instances the antigen will be present at levels about 75 times higher than other seed material. In some instances the antigen will be present at levels about 100 times higher than other seed material.

In some instances the antigen is combined with a pharmaceutically acceptable carrier for nasal administration. In some instances the antigen is administered nasally in the form of a nasal spray.

Where the vaccine is administered orally an antigen dose of between about 0.5 mg and 400 mg/kg body weight will in some instances be desirable. In some Instances it will be desirable to split the per-treatment antigen dose between several mucosal sites. In some instances a total per-treatment antigen dose of between about 1 mg and 300 mg/kg body weight will be desired. In some instances a total per-treatment antigen dose of between about 5 and 250 mg/kg body weight will be desired. In some instances, a series of 2 to 5 treatments, separated by between about 1 to 6 weeks will be desired. In some instances it will be desired to administer the antigen together with an immunogen. When this is done, it will in some instances be desired to reduce the antigen dose. One example of an immunogen is cholera toxin (“CT”).

It will be appreciated by those skilled in the art that doses can be readily determined with reference to the nature of the patient, its weight, and condition as well as the factors discussed herein. A suitable antigen dose for a particular route of administration and patient can be readily determined by administering an antigen dose within the ranges suggested above and monitoring the patient for serious adverse effects (such as anaphylactic shock) and for the generation of specific antibodies recognizing the antigen when administered. A suitable dose is any dose which does not cause serious adverse effects and which induces the generation of clinically useful levels of specific antibodies recognizing the antigen when administered over a course of treatment involving one or more instances of antigen administration over a period of between about 2 weeks to 6 months.

Production of viral antigens in plant seeds is desirable for the reasons previously discussed. While antigen expression need not be limited to seed tissue, it is preferred to have high levels of antigen expression in seeds. In some instances, promoter sequences allowing preferential expression of the regulated gene in seed tissue will be desired. In some instances, promoters which are substantially specific for gene expression in seeds (causing little or no expression of the antigen in unrelated tissues) will be preferred.

Promoters will generally be selected for effectiveness in the plant species used for expression. In some instances, it will be desirable to use promoter sequences derived from those occurring naturally in the plant variety employed or from a member of the same plant family. In some instances, where expression in monocotolydonous plants is desired, endosperm specific seed storage protein promoters will be desired. For example, promoters selected from the glutelin family such as gt1, gt2 and gt3 will be desirable in some instances. In some instances it will be desired to use a promoter from the 11S-12S seed storage gene family, such as a member of the family of the glycins, legumins or globulins. In some instances a promoter from the oat globulin gene family will be desired, such as glav1 and glav 3.

Post-translational modification and targeting of the expressed viral antigen is governed, in part, by the signal sequence (s.s.) employed. Signal sequences will generally be selected based on their effectiveness in the plant species employed for antigen expression. In some instances, it will be desirable to use signal sequences derived from those occurring naturally in the plant variety employed or from a member of the same plant family. In some instances, it will be desired to use a signal sequence derived from the same or a similar source as the promoter sequence employed. Where seed-specific expression is desired, it will sometimes be desired to use a signal sequence from an endosperm specific seed storage protein. In some instances it will be desired to use a signal sequence from 11S-12S seed storage family. In some instances, it will be desired to use a signal sequence targeting the nascent protein to the endoplasmic reticulum, then to the Golgi Apparatus, and then to protein storage vacuoles/vesicles (PSV). Previous reports have shown difficulties in successfully targeting foreign proteins to PSV's. The present invention overcomes these challenges and provides a method for expressing foreign proteins in cereal crop PSV's.

In some instances it will be desired to express a heterologous fusion protein containing a herpes virus antigen together with one or more additional heterologous protein sequences. Such additional heterologous sequences may be employed to facilitate enrichment or purification of the antigen (for example through the use of a c-myc or polyhistidine tag), to increase antigen yields (for example by adding a endoplasmic reticulum retention signal such as HDEL), or to increase immunogenicity (for example by adding a protein or peptide sequence having adjuvant activity such as cholera toxin or E. coli LT toxin.

The use of plant seeds for vaccine production offers several advantages. Particulate matter, such as whole or ground seed material, is immunostimulatory, thereby allowing forenhanced vaccine effectiveness when the antigen is not entirely separated from seed material prior to administration. Moreover, by targeting antigen expression to storage vacuoles in endosperm tissue (such as PSV's), the antigen can be produced in an environment where it can be stable without refrigeration for long periods of time (if kept reasonably dry (less than 45% relative humidity and preferably less than 25% relative humidity) and, preferably, below about 40° C.). In some instances the seed is stored in the dark or under low light conditions. However, good stability is observed under day light conditions as well. In some instances, the antigen produced according to the method of the invention is stable for at least 4 months. In some instances, the antigen produced according to the method of the invention is stable for at least 8 months. In some instances, the antigen produced according to the method of the invention is stable for at least 12 months. In some instances, the antigen produced according to the method of the invention is stable for at least 2 years. In some instances, the antigen produced according to the method of the invention is stable for at least 4 years.

In some instances, it will be desirable to optimize the nucleotide sequence encoding the antigen of interest for efficient expression in the seeds of the selected plant. In light of the disclosure herein, one skilled in the art could readily select codons encoding the same amino acids as found in the native antigenic protein but subject to more efficient expression in seeds of the selected plant.

It will be apparent to one skilled in the art that small in-frame mutations may be made to the nucleotide sequence encoding the antigenic protein or peptide of interest while retaining antigenic function. Such mutations are preferably either located outside of the region encoding known major antigenic regions of interest or are conservative mutations. In some instances the mutant sequence will be at least 80% homologous to the typical native sequence. In some instances the mutant sequence will be at least 90% homologous to the typical native sequence.

In some instances, it will be desirable to introduce a nucleic acid encoding a herpes virus antigen of interest by transforming cereal plant tissue which is competent to form seeds.

As used herein, the phrase “cereal plant tissue” means plant seeds, parts, tissue or cells substantially derived from a cereal plant. As used herein, the phrase “competent to form seeds” means able, in the course of development according to methods known on the art and/or disclosed herein, to form plant seeds.

In some instances, it will be desirable to transform cereal plant tissue using a suitable strain of Agrobacteria. As used herein, the phrase “suitable strain of Agrobacteria” means a strain of Agrobacteria which is capable of being transformed to contain a nucleic acid encoding a herpes virus antigen, and of transforming the cereal plant of interest to introduce into that plant the nucleic acid encoding the herpes virus antigen in expressible form.

While cereal plant tissue may be conveniently transformed using Agrobacteria, other transformation methods will be appropriate in some instances. For example, in some instances it will be desirable to transform cereal plant tissue competent to form seeds using a biolistic gun.

### EXAMPLE 1

### Construction of Core Plasmid Vector pPH2/gB/NOS-T

The plasmid pPH2/gB/NOS-T was designed so that transcription of the full Towne strain gB sequence (SEQ. ID. NO. 2) of approximately 2.7 kb was directed by the glutelin Gt3 promoter (SEQ. ID. NO. 1). In addition, to facilitate targeting of gB polypeptides to the secretory pathway of plant cells, the 72 bp signal sequence of gB was removed and replaced with the 72 bp signal sequence (“s.s.”) of Gt3 (SEQ. ID. NO. 3).

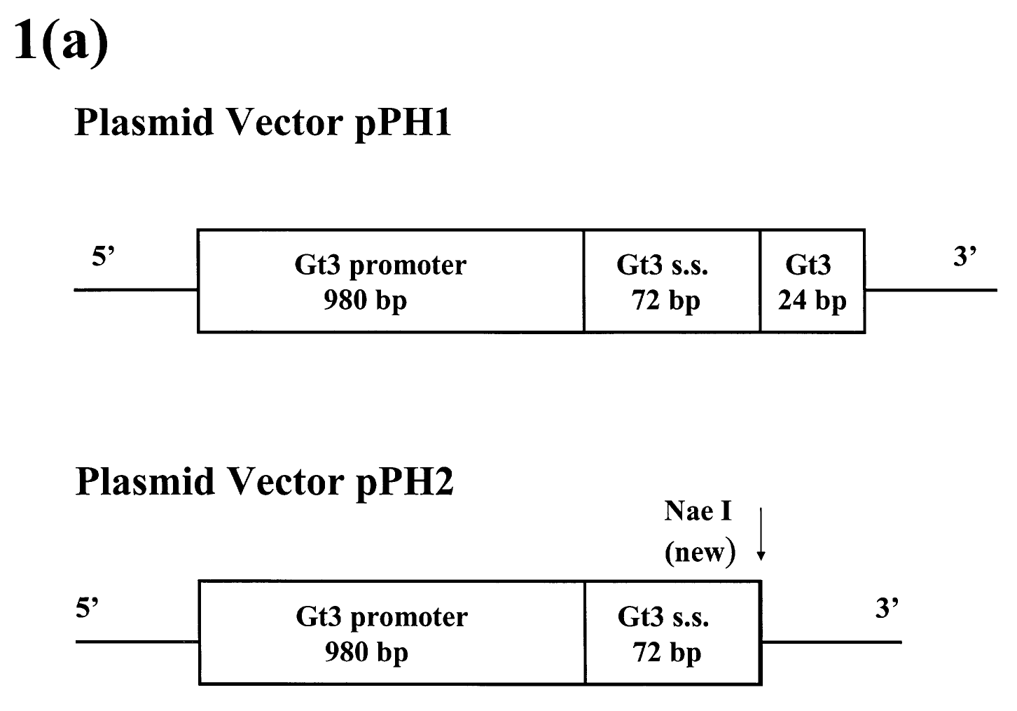

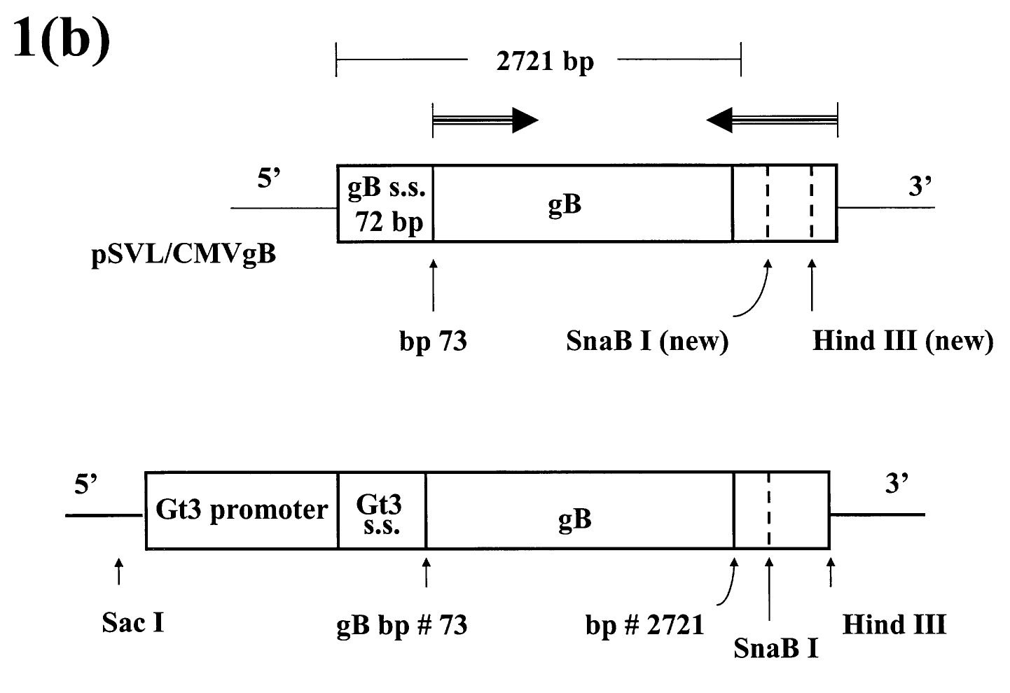

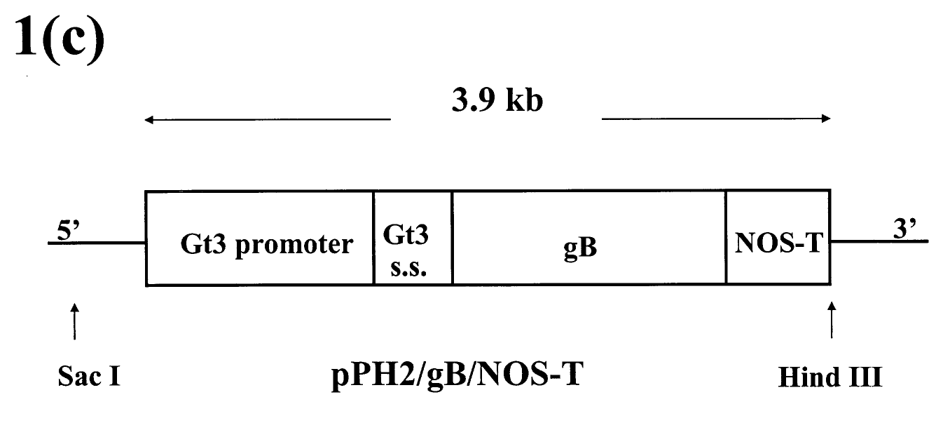

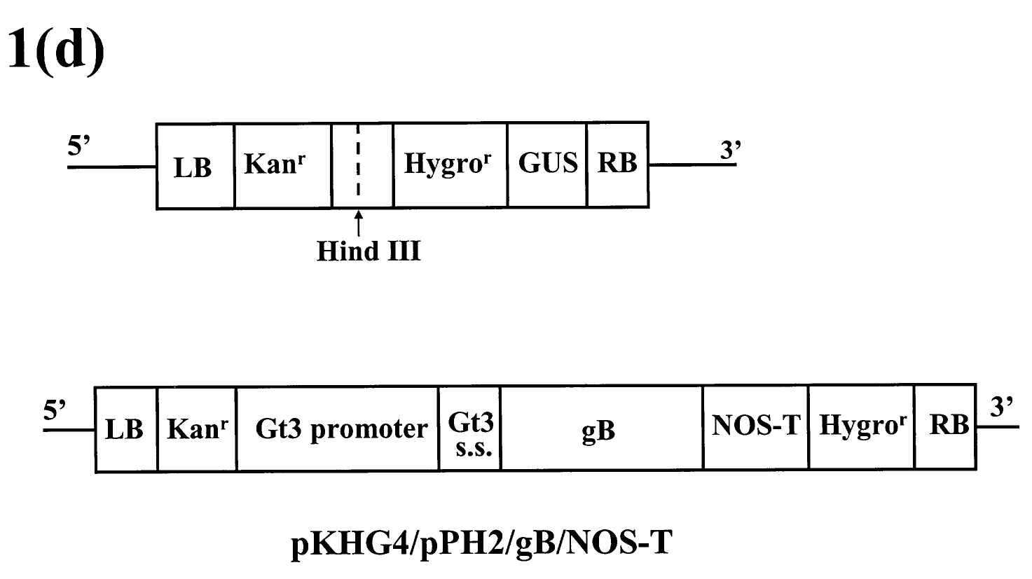

The insert structure of the plasmid expression vector pPH2/gB/NOS-T is shown in FIG. 1(c). This vector was prepared substantially as described in Tackaberry et al. (1999), above. Unless specified otherwise, before each subcloning step the DNA was purified by Gene Clean™ (Bio101™, La Jolla, Calif.) and, when necessary, concentrated by precipitation with ethanol after phenol-chloroform extraction. Ligation reactions, transformation of E. coli (DH5-a or STBL2, Canadian Life Technologies™, Burlington, ON) and selection of recombinant colonies with appropriate antibiotic were all performed using standard protocols. DNA sequencing was carried out on ligation products to insure correct orientation and fidelity of ligation junctions.

In brief, the plasmid vector pPH1 (Ganz et al., Transgenic Plants, Owen ed., John Wiley & Sons, NY, 1996, p. 281; Sardana et al. Methods in Biotechnology, Vol. 3, Humana Press, N.J., 1997, p. 77)) was redesigned to allow ligation of the Gt3 signal sequence (SEQ. ID. NO. 3) adjacent to cDNA encoding the mature gB protein, without N-terminal sequences of the mature glutelin protein. This was accomplished by PCR amplification using customized primers to introduce a new and unique Nae I site, and subsequent subcloning of the PCR product into plasmid pGEM-4Z (Promega™, Madison, Wis.). The new plasmid was designated pPH2 (FIG. 1a). The gB sequence of the prototype HCMV Towne strain is encoded in an open reading frame of 2721 bp and includes at its 5′ terminus 72 bp coding for a signal peptide which is not present in the mature protein. The coding sequence for the entire mature gB protein of 907 amino acids was amplified and then subcloned in-frame as a blunt end-Hind III fragment into pPH2, yielding pPH2/gB (FIG. 1b). Thereafter, DNA (SEQ. ID. NO.4) encoding NOS-T, a termination signal derived from the nopaline synthase gene, was amplified from pCaMVCN (Pharmacia Biotech™, Montreal, Que.) and subcloned as a SnaB 1-Hind III fragment into pPH2/gB. The resulting construct was called pPH2/gB/NOS-T (FIG. 1c).

### EXAMPLE 2

### Rice Transformation, Culture, Regeneration and Growth

To enable rice transformation and subsequent selection of transformants, pPH2/gB/NOS-T, was subcloned as a Hind III-Hind III fragment into pKHG4, a binary vector derived from pBin 19 and carrying the hygromycin phosphotransferase gene (which confers hygromycin resistance), the NPTII gene conferring kanamycin resistance, and the beta-D-glucuronidase (GUS) gene (FIG. 1d) (pKHG4 is described in LeGall et al., Plant Sci. 102, 161-170 (1994).). Recombinant plasmids were selected by resistance to kanamycin and were then used to transform Agrobacterium tumefaciens (strain EHA105), which were also selected by kanamycin resistance.

Subsequently, A. tumefaciens carrying plasmid pKHG4/pPH2/gB/NOS-T was used to transform rice callus that had been induced from seeds (substantially according to the method of Cheng et al. (Methods in Biotechnology, Vol. 3, Humana Press Inc., N.J., 1997, p. 1)). Maintenance of calli and selection with hygromycin were carried out as described in Cheng (1997), above. Once roots developed, rice seedlings were grown to maturity in soil in a controlled chamber at 28° C. and 50-60% relative humidity. Young leaves for PCR and Southern blotting analyses were collected and stored at −85° C. and mature seeds for ELISA analysis were collected approximately 5 weeks post pollination and stored at −20° C.

### EXAMPLE 3

### PCR Analysis for the Transgene

Genomic DNA was extracted from leaves of regenerated rice plants obtained according to Example 2 (Easy-DNA KitT™, Invitrogen™). Five hundred to 1000 ng of each sample was used as template in PCR reactions of 75 μL utilizing primers corresponding to the sequence at the 5′ end of the gene encoding the mature gB protein, and a downstream sequence within the gB gene, such that amplification would yield a PCR product of 1.2 kb. A positive control (plasmid containing pPH2/gB/NOS-T) and negative controls (DNA from non-transformed rice leaves, and a reaction mixture lacking template DNA) were included with each experiment. Analysis of the PCR products was carried out by loading each sample onto a 0.8% agarose gel containing ethidium bromide, followed by electrophoresis and visualization via ultraviolet transillumination. A molecular weight standard (1 kb ladder, Canadian Life Technologies™) was included with each experiment.

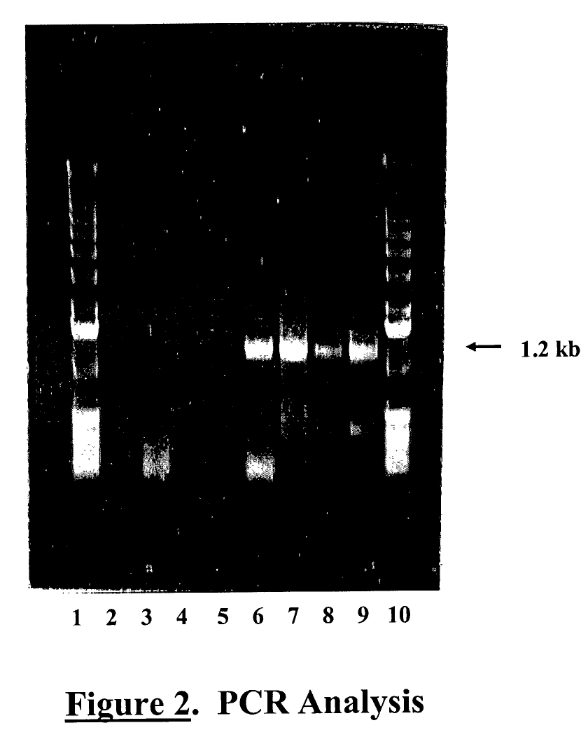

In FIG. 2, Lanes 1 and 10 contain molecular weight standard, 1 kb ladder; lane 3, negative control, no template DNA; lane 4, negative control, DNA from non-transformed rice; lane 6, gB-1; lane 7, gB-2; lane 8, gB-3; lane 9, gB-4.

Results of PCR analysis showed that 6 of 10 tested transformed plants were positive (i.e., carried the gB transgene) compared to the non-transformed negative controls. These plants were numbered gB-1, gB-2, gB-3, gB-4, etc. FIG. 2 shows representative PCR products following electrophoresis. No bands at 1.2 kb are seen in lanes 3 (negative control, no template); nor in lane 4, with, DNA from non-transformed rice. Lanes 6-9 are all positive for this band, demonstrating that gB-1, -2, -3 and -4 all contain this portion of the gB gene construct.

### EXAMPLE 4

### Southern Blot Analysis of the Transgene

Southern blot analysis was carried out on genomic DNA from 2 PCR-positive plants (gB-1 and gB-2). Ten μg of each genomic DNA sample was digested with Hind III, electrophoresed through 0.8% agarose and then alkali blotted onto a Pall Biodyne B™ membrane (Canadian Life Technologie™). Pre-hybridization (QuickHyb™, Stratagene™, La Jolla, Calif.) was followed by DNA hybridization using a random primed ³²P-dATP labelled probe generated from the pPH2/gB/NOS-T construct. Washings were carried out according to manufacturer's directions and the membrane then exposed to Kodak XOMAT XAR film with 2 intensifying screens at −85° C.

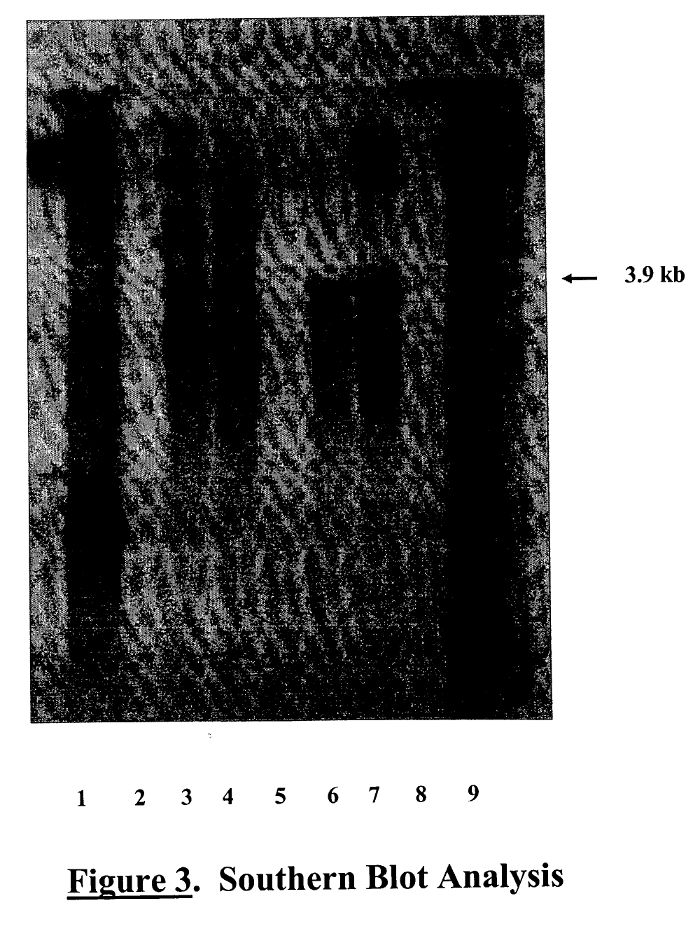

In FIG. 3, lanes 1 and 9 contain 1 kb ladder; lanes 3 and 4, positive control, plasmid pPH2/gB/NOS-T; lane 5, DNA extracted from non-transformed rice leaves; lane 6, DNA extracted from gB-1; lane 7, DNA extracted from gB-2.

Results of Southern blotting analysis are shown in FIG. 3. The data demonstrate that both gB-1 and gB-2 rice samples, in lanes 6 and 7, respectively, exhibited hybridizing fragments corresponding to the 3.9 kb transgene, as expected, confirming the PCR data. DNA extracted from non-transformed control rice (lane 5) did not reveal any hybridizing fragments. The data provide some evidence of transgene heterogeneity in gB-1, with restriction fragments of two sizes identified by a probe specific for sequences of pPH2/gB/NOS-T. Such multiple hybridization fragments may result from tandem inserts, elimination of restriction sites flanking pPH2/gB/NOS-T, rearrangement or partial deletion of the insert, or scrambling of transferred DNA by recombination events during or after insertion. In light of the disclosure herein, such issues can be identified (by screening for nucleotide insert and protein product size) and resolved (by selecting plants having the desired insert and product) routinely prior to the large-scale production of transformed plants for vaccine production.

### EXAMPLE 5

### Protein Extraction from Seeds for ELISA Measurement of gB

To extract soluble proteins from the rice seeds, mature rice seeds were manually dehusked, ground finely and mixed with ice cold extraction buffer [(20 mM Tris-HCl pH 7.4 containing 0.5M NaCl, 0.1% (v/v) Triton X-100™, 1% (w/v) ascorbic acid, 1% (w/v) insoluble polyvinylpolpyrrolidone (PVP), 1× complete protease inhibitor (Boeringher Mannheim™)], then briefly set on ice. The ground seeds were then centrifuged and the resulting supernatant further clarified by centrifugation. The resulting extract was frozen in liquid N₂ and stored at −85° C.

### EXAMPLE 6

### gB-Specific ELISA (Enzyme Linked Immunosorbant Assay)

Extracts from plant gB-2, obtained according to Example 6 were examined by ELISA. Volumes of 50 μL were used throughout the assay unless otherwise specified, and wells were washed between each step with 3×300 μL of wash buffer (PBS pH 7.2 containing 0.02% Tween 20™ and 0.02% NaN₃). Wells of Immulon II™ microtiter plates (Dynatech Laboratories™) were coated with 23 μg/mL of the F(ab′)₂ fragment of CMVB1, a gB-specific murine monoclonal antibody (mAb) with viral neutralizing activity against HCMV in phosphate buffered saline (“PBS”) pH 7.2. After overnight incubation at room temperature the wells were blocked with 2% (w/v) bovine serum albumin (BSA) in PBS for 2 h at 37° C., followed by addition of test sample or control for 1 h at 37° C.

Prior to assay the test samples (seed extracts) were adjusted so that they were at comparable protein concentrations (BioRad Protein Assay™, Hercules, Calif.) and none was greaterthan that of the control non-transformed seed extracts.

A pool of gB-specific monoclonal antibodies (mAb) (CMVB1 as above, ABI #127 and #128, ABI Inc., Columbia, Md.) diluted 1/20 in 2% BSA/PBS was used as the primary antibody and was added to the wells at 37° C. for 1 h. Fc-specific goat anti-mouse IgG conjugated to alkaline phosphatase (Jackson Laboratories™, Westgrove, Pa. and ICN Biomedicals™ Inc., Aurora, Ohio), diluted 1/10,000 in 3% BSA/PBS was then added and incubated at 37° C. for 1 h. This was followed by addition of phosphatase substrate (1 mg/mL p-nitrophenyl phosphate, Sigma™, St. Louis, Mo.) in 10% diethanolamine pH 9.6, for 60 min, after which absorbance at 405 nm was measured spectrophotometrically (Dynatech MRX™ Plate Reader). Sample wells were blanked against wells in which buffer was substituted for test sample and all assays were performed in triplicate.

Raw ELISA data were converted to ng gB/mg total extracted protein by reference to an ELISA standard curve constructed with recombinant gB produced in Chinese hamster ovary (CHO) cells (Austral Biologics™, San Ramon, Calif.). (Tackaberry et al. Vaccine 17:3020(1999)).

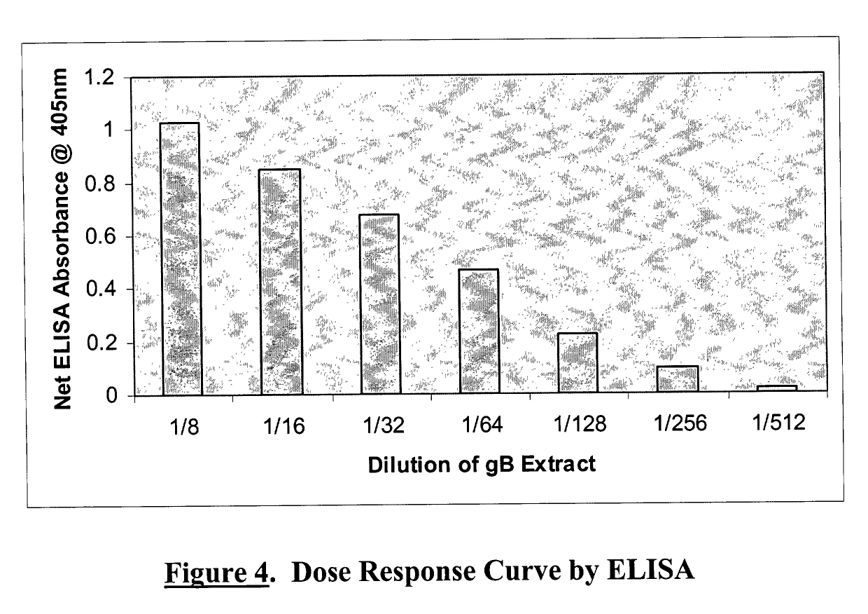

ELISA results of gB-2 were positive, compared to an extract of non-transformed rice seeds at comparable protein concentration. As shown in FIG. 4, the dose-response curve for dilutions of seeds extract is linear between dilutions of 1:8 and 1:256, confirming the specificity of the ELISA response. By reference to the standard curve, the concentration was calculated as 4.7 μg gB/mg total seed protein, representing approximately 0.5% of total seed protein. These data show that gB expression was successfully targeted to the seeds of transformed rice plants. The recombinant protein synthesized in the seeds retained the neutralizing CMVB1 epitope(s) of the wild-type molecule (the target epitope of the monoclonal antibody CMVBI). It is probable that only about 50% of the recombinant protein (gB or GM-CSF) was extracted by the procedure employed. Thus, the amount of gB actually produced was likely about 2-fold greater.—ie, as high as 1% of total seed protein. Once homozygous plant lines are developed, the level of specific expression often increases substantially over that recovered in the seeds of primary transformants. After 8 months of storage at 4° C. in a dry environment, the same gB-2 seeds were retested by ELISA. Results were comparable to the original data, demonstrating that the recombinant gB was quite stable under these conditions.

### EXAMPLE 7

### Western Blotting

SDS-PAGE was performed with a Bio-Rad™ minigel apparatus and the discontinuous system of Laemmli (Nature 227:680 (1970)), with a final acrylamide monomer concentration in the separating gel of 7.5% (w/v). The gel was transferred to BioRad™ PVDF membrane using the BioRad Miniblot™ system and electrophoresing at 100V for 1.5 h. After blocking (Tris-buffered saline with 0.05% Tween-20 (TBST) containing 3% skim milk powder for 1 h at room temperature), the membrane was incubated overnight at 4° C. in a mixture of monoclonal antibodies specific for HCMV gB (CMVBI, (Tackaberry et al., J. Virol. 67:6815 (1993)); CMM-1401-5, (Austral Biologicals™, San Ramon, Calif.); 13-127-100 and 13-128-100 (Advanced Biotechnologies™ Inc., Columbia, Md.; diluted 1/33 in blocking buffer)). The membrane was subsequently washed with TBST, incubated with goat-anti-mouse IgG conjugated to alkaline phosphatase (AP), washed again and then developed calorimetrically using the BioRad AP substrate kit.

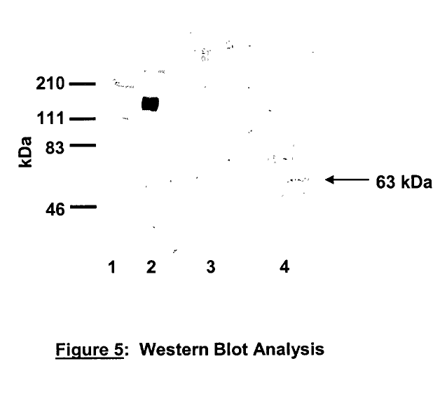

In FIG. 5, the lanes contain the following: Lane 1: Biorad Prestained High Molecular Weight Markers; Lane 2: 100 ng of recombinant HCMV gB from CHO cells, 140 kDa; Lane 3: 79 μg (total protein) of non-transformed (control) rice seed extract; Lane 4: 117 μg (total protein) of gB-2 transgenic rice seed extract. The arrow indicates the transgenic rice gB at 63 kDa.

The Western blot shown in FIG. 5 reveals one unique band in the sample derived from the seeds of gB-2 (lane 4). Our calculations indicate that the size of this band is 63 kDa. This is slightly larger than the 55-58 kDa of the human subunit. The most likely explanation for the variance in size is that there are differences in glycosylation between the rice-derived gB and the native gB (ie, gB produced in human cells infected with HCMV).

The apparent absence of a band at 130-150 kDa (the unproteolyzed, precursor gB), or of bands between 93-116 kDa (glycoforms of the other subunit, gp116) suggests that the pool of 4 different monoclonal antibodies used (all gB specific) did not target epitopes on this part of the gB molecule. The apparent absence of the precursor 130-150 kDa band, from which the gp55 (here 63 kDa) is derived, may be due to very low concentration in the rice seeds (below the level of detection in this system), or may reflect a high sensitivity to degradation—either in the plant cells themselves, or during the protein extraction process. Although there was approximately 50% more protein loaded into lane 4 (gB-2 rice seed extract), compared to lane 3 (non-transformed rice seed extract), it is nonetheless clear that the absence of the putative gB band of 63 kDa in the control lane is not simply the result of this discrepancy.

### EXAMPLE 8

### Antigenicity of Transgenic gB from Rice Administered by Injection

Soluble Antigen

Recombinant gB produced as described In Example 2 and extracted substantially as described in Example 5 is injected into rabbits.

Recombinant gB antigen is injected at 3 sites for each immunization, (1 subcutaneous (sc) site in a volume of 0.1 mL, and 2 sites intramuscularly (im) with a volume of 0.2 mL each site). The antigen mixture actually prepared 1:1 (vol:vol) with a Freund's adjuvant or RIBI adjuvant (Cedarlane Laboratories). The soluble antigen is prepared at a concentration so that the total dose (per immunization at all 3 sites) is a consistent amount between 500-800 micrograms.

A second Immunization (ie, a booster injection) is carried out at 28 days; and if necessary (eg. If there is not any or not very much antibody detected), each 28 days for several months. Typically, trial bleeds are obtained at 2, 3 and 4 (=28 days) weeks after the first immunization; and then at 1 week after each boost Anti-gB antibody titer from these bleeds is assessed by standard methods.

Control animals receive injections of protein extracted from non-transformed rice.

Rabbits which received the recombinant gB by injection show significant levels of immune response to native gB, whereas control animals do not.

Thus, recombinant gB produced as described herein is useful as a vaccine against gB and HCMV when administered by injection.

It will be appreciated that cytomegaloviruses are generally species specific. Thus, when protection from naturally-occurring infection is desired, a cereal crop expressing an antigen (such as gB) from a the cytomegalovirus naturally infecting the patient's species will be useful as a vaccine.

Particulate Antigen/Seed Material

Whole rice seeds from gB-expression plants are obtained and finely ground to provide ground seed particles. The immunization schedule described for soluble antigen is employed, but there are no intramuscular injections and no adjuvant is used (as the particulate seed material itself is highly immunogenic). Injection occurs at 2 sc sites, in a volume of 0.1 mL.

Rabbits which received the recombinant gB (in ground seeds) by injection show significant levels of immune response to native gB, whereas control animals do not

### EXAMPLE 9

### Antigenicity of Transgenic gB from Rice Administered to a Mucous Membrane

Transgenic gB in rice seeds Is produced as described in Example 2. The resultant rice seeds are fed to rabbits, guinea pigs, cattle and swine at a level of 250 g/kg body weight on days 1, 30 and 60. gB receiving animals are split into three groups: those receiving whole seeds; those receiving coarsely ground seeds; and those receiving finely ground seeds. Control animals receive untransformed seeds.

One week after the last feeding of gB-containing-rice, the animals are bled and serum Is tested for reactivity to native gB.

Rabbits, guinea pigs, cattle and swine which received the recombinant gB by oral administration in their feed show significant levels of immune response to native gB. A significant immune response is observed from all groups of gB-receving animals, regardless of whether the seed is whole, coarsely ground or finely ground.

Thus, recombinant gB produced as described herein is useful as a vaccine against gB when administered orally.

### EXAMPLE 10

### Antigen Storage Stability

Mature rice seeds containing gB obtained from plants produced according to Example 2 are stored in dosed containers at either ambient U.S. laboratory humidity or at 45% relative humidity. Containers from each humidity group are stored at −85° C., −60° C., −40° C., −20° C., 0° C. 10° C. 20° C., 30° C. and 40° C. The stability of gB in rice stored under each set of conditions is assayed by ELISA at the start of the study, at months 3, 6, 9, 12, 15, 18, 21, 24, 30, 36, 42, 48, 54 and 60. No significant reduction in gB is observed in any of the samples for a 2 year period. Only small (<20%) reductions in gB levels are observed in samples stored for 2-4 years. Reduction of gB levels of less than 30% are observed for samples stored for 5 years. Thus, gB containing rice seeds of the present invention provides a very stable means for vaccine storage.

Thus, there has been provided a method for the production of a viral antigen in plant material which is a major food substance in a mammalian diet that fully satisfies the objects, aims and advantages set forth above. While the invention has been described in conjunction with specific embodiments thereof, it is evident that many alternatives, modifications and variations will be apparent to those skilled in the art in light of the foregoing description. Accordingly, it is intended to embrace all such alternatives, modifications and variations as fall within the spirit and broad scope of the invention.

What we claim as our invention:

1. A method of producing a herpes virus vaccine in the seeds of a cereal crop, said method comprising:
a) obtaining a nucleic acid sequence encoding a herpes virus antigen;
b) introducing the nucleic acid sequence into cereal plant tissue competent to form seeds;
c) permitting said cereal plant tissue to develop; and,
d) directing preferential expression of the antigen encoded by the nucleic acid sequence in seeds formed by the cereal plant tissue.

2. The method of claim 1 wherein the herpes virus is human cytomegalovirus.

3. The method of claim 1 wherein the cereal crop is selected from the group consisting of rice, wheat, oats, barley, and corn.

4. The method of claim 3 wherein the cereal crop is selected from the group consisting of rice, wheat, oats and corn.

5. The method of claim 1 wherein the cereal crop is rice.

6. The method of claim 1 wherein the herpes virus antigen is naturally expressed by at least one of human cytomegalovirus, herpes sixplex virus-1, herpes simplex virus-2, Epstein Barr virus, or varicello-zoster virus-8.

7. The method of claim 1 wherein the antigen comprises substantially the entire mature form of an antigenic herpes virus protein.

8. The method of claim 1 wherein the antigen is an envelope glycoprotein.

9. The method of claim 1 wherein the antigen comprises one or more antigenic domains selected from an antigenic herpes virus protein.

10. The method of claim 1 wherein the antigen comprises at least one or more antigenic domains of gB, gH or gD.

11. The method of claim 1 wherein the antigen comprises one or more antigenic domains of herpes glycoprotein B complex (“gB”).

12. The method of claim 11 wherein the antigen is selected from at least one of AD-1, AD-2 and AD-3.

13. The method of claim 1 wherein the antigen comprises a heterologous fusion protein.

14. The method of claim 1 wherein the nucleic acid sequence is cDNA.

15. The method of claim 1 wherein the nucleic acid is introduced by transformation.

16. The method of claim 15 wherein transformation is effected using a suitable strain of Agrobacterium.

17. The method of claim 1 wherein the cereal plant tissue is callus tissue.

18. The method of claim 1 wherein expression of the antigen in seeds is directed using a gene promoter which directs high levels of expression in seeds.

19. The method of claim 1 wherein expression of the antigen in mature seeds is at least twice as high as in other portions of the mature plant.

20. The method of claim 19 wherein the gene promoter is substantially seed specific.

21. The method of claim 19 wherein the gene promoter is an endosperm specific seed storage protein promoter.

22. The method of claim 21 wherein the gene promoter is a promoter from the glutelin family.

23. The method of claim 22 wherein the gene promoter is selected from gt-1 and gt-3.

24. The method of claim 19 wherein the gene promoter is a 11S-12S seed storage promoter.

25. The method of claim 24 wherein the promoter is selected from the group consisting of: glycin promoters, legumin promoters, and globulin promoters.

26. The method of claim 25 wherein the promoter is selected from glav1 or glav3.

27. A cereal crop seed containing a herpes virus antigen.

28. The seed of claim 27 produced according to the method of claim 1.

29. The seed of claim 27 wherein the antigen is stable when stored at no more than 45% relative humidity at a temperature between 0° C. and 40° C. for at least 12 months.

30. The seed of claim 27 wherein the cereal crop is selected from the group consisting of rice, wheat, oats, barley, and corn.

31. The seed of claim 27 wherein the cereal crop is rice.

32. The seed of claim 27 wherein the herpes virus is human cytomegalovirus.

33. The seed of claim 27 wherein the antigen comprises at least one human cytomegalovirus glycoprotein B complex (“gB”) antigenic region.

34. The seed of claim 33 wherein the antigen is substantially the entire mature gB protein.

35. The seed of claim 33 wherein the antigen comprises at least one of AD-1, AD-2 or AD-3.

36. The seed of claim 27 wherein the antigen is a heterologous fusion protein.

37. A method of inducing an immune response in a mammal which response reduces the risk of subsequent infection of the mammal by a herpes virus, said method comprising administering to the mammal a herpes virus antigen obtained from cereal crop seeds of claim 27.

38. The method of claim 37 wherein the cereal crop is selected from at least one of rice, wheat, oats, barley, and corn.

39. The method of claim 37 wherein the cereal crop is rice.

40. The method of claim 37 wherein the herpes virus is human cytomegalovirus.

41. The method of claim 37 wherein the cereal crop seed is produced according to the method of claim 1.

42. The method of claim 37 wherein the antigen seed is administered to a mucous membrane.

43. The method of claim 37 wherein the antigen is administered orally.

44. The method of claim 37 wherein the antigen is administered nasally.

45. The method of claim 37 wherein the cereal crop seed containing the antigen is administered orally.

46. The method of claim 37 wherein the crop seed is administered substantially without enrichment or purification of the antigen.

47. The method of claim 37 wherein the antigen is enriched relative to other crop seed components prior to administration.

48. The method of claim 37 wherein the antigen is administered by injection.

49. A nucleic acid encoding: a signal peptide sequence directed to the endoplasmic reticulum, then the Golgi Apparatus, and then to protein storage vacuoles; a herpes virus antigen; and, a promoter sequence allowing preferential expression of a controlled gene in cereal crop seeds, wherein the portion of the nucleic acid encoding the signal sequence and the herpes virus antigen is the controlled gene.

50. The nucleic acid of claim 49 wherein the herpes virus is human cytomegalovirus.

51. The nucleic acid of claim 49 wherein the promoter is an endosperm specific seed storage promoter.

52. The nucleic acid of claim 49 wherein the promoter is a 11S-12S seed storage promoter.

53. The nucleic acid of claim 49 wherein the nucleic acid is DNA.

54. A cereal crop cell containing a stably incorporated nucleic acid encoding an antigenic domain of a herpes virus antigen.

55. The cell of claim 54 wherein the cereal crop is rice and the antigenic domain is a domain of gB.

56. A herpes virus vaccine produced according to the method of claim 1.

57. Agrobacterium tumefaciens transformed with the nucleic acid of claim 49.


SEQUENCE LISTING (4 sequences)

SEQ ID NO: 1 - DNA, 980 bp, Oryza sativa
  ref <301>: AUTHORS: Okita et. al.
  ref <302>: TITLE: Structure and Expression of the rice glutelin multigene family
  ref <303>: JOURNAL: J. Biol. Chem.
  ref <304>: VOLUME: 264
  ref <305>: ISSUE: 21
  ref <306>: PAGES: 12573-12581
  ref <307>: DATE: 1989
  ref <309>: DATABASE ENTRY DATE:
  ref <313>: RELEVANT RESIDUES: (1)..(980)
tctagaagaaagataaataaccgaaactatttggagagcattcaggttacatggttagtccatggtgctagatattgctatataatactcaatgcaatgctcaatagatataagtttcaaagctgtataagaattttaggttagtgtgcaatgtaagtgtagcttcttatagcttagtgctttactatcttcacaagcacatgctatagtattgttccaagatgaaagaataattcatccttgctaccaacttgcatgatattatatttgtgaatatcctatctcttggcttataatgaaatgtgctgctgggttattctgaccatggtatttgagagcctttgtatagctgaaaccaacgtatatcgagcatggaacagagaacaaaatgcaaggatttttttattctggttcatgccctggatgggttaatatcgtgatcatcaaaaaagatatgcataaaattaaagtaataaatttgctcataagaaaccaaaaccaaaagcacatatgtcctaaacaaactgcattttgtttgtcatgtagcaatacaagagataatatatgacgtggttatgacttattcactttttgtgactccaaaatgtagtaggtctaactgattgtttaaagtgatgtcttactgtagaagtttcatcccaaaagcaatcactaaagcaacacacacgtatagtccaccttcacgtaattctttgtggaagataacaagaaggctcactgaaaaataaaagcaaagaaaaggatatcaaacagaccattgtgcatcccattgatccttgtatgtctatttatctatcctccttttgtgtaccttacttctatctagtgagtcacttcatatgtggacattaacaaactctatcttaacatctagtcgatcactactttacttcactataaaaggaccaacatatatcatccatttctcacaaaagcattgagttcagtcccacaaaaac

SEQ ID NO: 2 - DNA, 4256 bp, Human Cytomegalovirus (Towne strain)
  ref <301>: AUTHORS: Spaete et. al.
  ref <302>: TITLE: Human cytomegalovirus strain Towne glycoprotein B is processed by proteolytic cleavage
  ref <303>: JOURNAL: Virology
  ref <304>: VOLUME: 167
  ref <305>: ISSUE: 1
  ref <306>: PAGES: 207-225
  ref <307>: DATE: 1988
  ref <309>: DATABASE ENTRY DATE:
  ref <313>: RELEVANT RESIDUES: (1)..(4256)
ctgcagaaggtttcggaggtgggctatggcaaggtgatccgttgcatcaaaacgcaggagcgtctgactagcaaactcatcgatgtcaacctagtggggcccttgtgtctggactttatctctaagctcatgaatgggtttctataccgcagccaataccaccaggaccaagacgtggtggacgtgggggatcagttcacgtacgatgagcacctgtatgtggtcaataacctgatccacaagagtctgcctgtggaatccctcccgctactgggtcagcagatctacgagttgtgtaacgggcccctcttcacacactgcaccgatcgctatcccctctctcacaatgtggacatggcctatgcctgcgacaacgcgggcgtactaccccacgtcaaggacgatttggtcaaatgcgcggaaggtaccgtgtatcccagtgagtggatggtggtgaagtacatgggttttttcaatttttcggactgtcaggatctaaacgtgctgcagaaggagatgtggatgcacgtgcgggagctcgtgctctccgtcgcactatataatgaaactttcgggaaacaactctcgatcgcgtgcctgcgcgacgaactgcacccggacagagatgtgattctcacgtataacaaagagtggccgctgctgcttcgtcacgaaggaagtctttataagtccaaagatctatatctcctcctctacaggcatctgtccagaccggatgagagtggcgacgtgccaacagctcccgcggccaagccctccaccctgacggccgccgcggccgtctcgggtgccttcagggagccggaccgaccttggctgccaagtccgtacccctcctcctcgaccgcgggcgtttcccggagggtccgcgcaacacgcaagagaccacgacgcgcctcatcgctgctggatttggcccgcgacgaacatggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcattctcgatccggttcagtctctcaacgcgtaacttcttcccaaacggtcagccatggtgttaacgagaccatctacaacactaccctcaagtacggagatgtggtgggggtcaacaccaccaagtacccctatcgcgtgtgttctatggcacagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctaccagaaggttttgacgtttcgtcgtagctacgcttacatccacaccacttatctgctgggcagcaacacggaatacgtggcgcctcctatgtgggagattcatcatatcaacagtcacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgagacctgtaatctgaattgtatggtgaccatcactactgcgcgctccaagtatccctatcattttttcgcaacttccacgggtgatgtggttgacatttctcctttctacaacggaactaatcgcaatgccagctattttggagaaaacgccgacaagtttttcatttttccgaactacactatcgtctccgactttggaagaccgaattctgcgttagagacccacaggttggtggcttttcttgaacgtgcggactcagtgatctcctgggatatacaggacgagaagaatgttacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgtgtacgtgatgaggccataaataagttacagcagattttcaatacttcatacaatcaaacatatgaaaaatatggaaacgtgtccgtctttgaaaccactggtggtttggtggtgttctggcaaggtatcaagcaaaaatctctggtggaactcgaacgtttggccaaccgctccagtctgaatcttactcataatagaaccaaaagaagtacagatggcaacaatgcaactcatttatccaacatggagtcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaacttagcaagatcaacccgtcagctattctctcggccatctacaacaaaccgattgccgcgcgtttcatgggtgatgtcctgggtctggccagctgcgtgaccattaaccaaaccagcgtcaaggtgctgcgtgatatgaatgtgaaggaatcgccaggacgctgctactcacgaccagtggtcatctttaatttcgccaacagctcgtacgtgcagtacggtcaactgggcgaggataacgaaatcctgttgggcaaccaccgcactgaggaatgtcagcttcccagcctcaagatcttcatcgccggcaactcggcctacgagtacgtggactacctcttcaaacgcatgattgacctcagcagcatctccaccgtcgacagcatgatcgccctagacatcgacccgctggaaaacaccgacttcagggtactggaactttactcgcagaaagaattgcgttccagcaacgtttttgatctcgaggagatcatgcgcgagttcaattcgtataagcagcgggtaaagtacgtggaggacaaggtagtcgacccgctgccgccctacctcaagggtctggacgacctcatgagcggcctgggcgccgcgggaaaggccgttggcgtagccattggggccgtgggtggcgcggtggcctccgtggtcgaaggcgttgccaccttcctcaaaaaccccttcggagccttcaccatcatcctcgtggccatagccgtcgtcattatcatttatttgatctatactcgacagcggcgtctctgcatgcagccgctgcagaacctctttccctatctggtgtccgccgacgggaccaccgtgacgtcgggcaacaccaaagacacgtcgttacaggctccgccttcctacgaggaaagtgtttataattctggtcgcaaaggaccgggaccaccgtcgtctgatgcatccacggcggctccgccttacaccaacgagcaggcttaccagatgcttctggccctggtccgtctggacgcagagcagcgagcgcagcagaacggtacagattctttggacggacagactggcacgcaggacaagggacagaagcccaacctgctagaccgactgcgacaccgcaaaaacggctaccgacacttgaaagactccgacgaagaagagaacgtctgaaccaagaggagaaaaaaaaactagacaaaaaaagtgacagagacttgtgatatacggggttaaactggacatctaggtgctgcatgtgtatttgctttgtgattttgcttcgtaagctgtcagcctctcacggtccgctatgtttttcaacccgtatctgagcggcggcgtgaccggcggtgcggttgcgggtggccggcgtcagcgttcgcagcccggctccgcgcagggctcgggcaagcggccgccacagaaacagtttttgcagatcgtgccgcgaggcgtcatgttcgacggtcagacggggttgatcaagcataagacgggacggttgcctctcatgttctatcgagagattaaacatttgttgagtcatgacatggtttggccgtgtccttggcgcgagaccctggtgggtcgcgtggtgggacctattcgttttcacacctacgatcaaacggacgccgtgctcttcttcgactcgcccgaaaacgtgtcgccgcgctatcgtcagcatctggtgccttcggggaacgtgttgcgtttcttcggggccacagaacacggctacagtatctgcgtc

SEQ ID NO: 3 - DNA, 72 bp, Oryza sativa
atggcaaccatcaaattccctatagttttctccgtcgtttgctttttcctcttgtgtaatggttcgttagcc

SEQ ID NO: 4 - DNA, 252 bp, pCaMVCN plasmid (Pharmacia/Amersham)
atcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc